(19) United States
(12) Patent Application Publication
(10) Pub. No.: US 2024/0117375 A1
(43) Pub. Date: Apr. 11, 2024

(54) GENETICALLY MODIFIED SORGHUM AND METHODS OF GENETICALLY ENGINEERING SORGHUM TO CONTROL CROSS-INCOMPATIBILITY

(71) Applicant: Board of Regents of the Nevada System of Higher Education on Behalf of the University of Nevada, Reno, NV (US)

(72) Inventor: Melinda Yerka, Reno, NV (US)

(21) Appl. No.: 18/367,282

(22) Filed: Sep. 12, 2023

Related U.S. Application Data
(60) Provisional application No. 63/406,098, filed on Sep. 13, 2022.

(51) Int. Cl. C12N 15/82; C12N 9/22; C12N 15/11

(52) U.S. Cl. CPC C12N 15/8287; C12N 9/22; C12N 15/111; C12N 15/8213; C12N 2310/20; C12N 2800/80

(57) ABSTRACT

The present disclosure provides genetically edited or modified plants of the genus Sorghum and methods of producing genetically edited or modified plants of the genus Sorghum using, e.g., a CRISPR/Cas9 gene editing tool. In some embodiments, the genetic modifications are directed to genes of the Inhibition of Alien Pollen (IAP) locus to modulate cross-compatibility of the plants with other species of Sorghum.

### CROSS-REFERENCE TO RELATED APPLICATION

This application claims the benefit under 35 USC § 119(e) of U.S. Provisional Application No. 63/406,098 filed Sep. 13, 2022, which is incorporated herein by reference in its entirety for all purposes.

### ACKNOWLEDGMENT OF GOVERNMENT SUPPORT

The invention was made with government support under grants 2017-33522-27086 and 2023-67014-39538 awarded by the National Institute of Food and Agriculture under United States Department of Agriculture. The government has certain rights in the invention.

### REFERENCE TO A SEQUENCE LISTING

This application incorporates by reference a computer readable Sequence Listing in ST.26 XML format, titled UNR22-045US_Sequence, created on Sep. 8, 2023 and containing 50,632 bytes.

### FIELD OF THE INVENTION

The present invention relates generally to genetically modified or edited plants of the genus Sorghum, and to methods of producing such genetically modified or edited plants.

### BACKGROUND

Sorghum bicolor is a C4 grain crop in the grass family Poaceae. It is related to other members of sub-family Panicoideae, including the staple crops maize (Zea mays L.) and rice (Oryza sativa L.), and is the 5^{th} most produced cereal crop in the world (Dahlberg, et al., 2011, CREA Journals 56, 2). The United States leads production of Sorghum bicolor globally. The genus Sorghum contains 25 species within five clades: Eu-sorghum, Heterosorghum, Parasorghum, Stiposorghum, and Chaetosorghum (Lazarides, et al., 1991, Aust Syst Bot 4, 591-635; Hodnett, G. 2005, Crop Science 45, 1403-1409). The S. bicolor genome has been assembled (Paterson, 2009, Nature 456-551) but is not adequately annotated.

S. bicolor has potential as a climate-resilient bioenergy crop and is nutritionally important as a gluten-free grain. However, S. bicolor's natural molecular mechanisms controlling cross-compatibility with the plant's relatives has hindered biotechnological and breeding developments. S. bicolor is cross-compatible with close relatives, such as the noxious weed Johnsongrass (Sorghum halepense), which limits biotechnological development due to risk of transgenes escaping into weed gene pools. Meanwhile, S. bicolor is not cross-compatible with distant relatives, such as Sorghum plumosum, which also limits biotechnological development and breeding endeavors of ingressing additional stress-tolerance genes into sorghum cultivars. Some aspects of the molecular mechanisms directing cross-compatibility in S. bicolor are known. For example, it is understood that the dominant wild-type allele of the IAP locus of S. bicolor prevents distant relatives' pollen tube growth into S. bicolor pistils and confers cross-incompatibility. However, the number, identity, and functions of the genes in the IAP locus are unknown.

### BRIEF SUMMARY OF THE INVENTION

Disclosed herein are genetically edited or genetically modified sorghum plants and methods of producing the GE or modified sorghum using, e.g., a CRISPR/Cas9 gene editing system. According to embodiments of the present disclosure, novel engineered cross-incompatibility systems are disclosed which, when genetically edited, enable cross-compatibility between Sorghum species that would otherwise not be possible. In some cases, gene flow can be improved between Sorghum species and distant relatives with desirable stress-tolerance traits.

In one aspect, the present disclosure provides a genetically modified plant of the genus Sorghum, comprising a modification of one or more genes contained in an inhibition of alien pollen (IAP) locus of the plant, wherein the one or more genes controls cross-incompatibility of the plant with other species of Sorghum. In some cases, the genetically modified plant is of the species Sorghum bicolor.

In some embodiments, one or more genes in the IAP locus are selected from the group consisting of Sobic.002G023300, Sobic.002G023400, Sobic.002G023500, and Sobic.002G023600, and Sobic.002G023700. In some cases, the one or more genes in the IAP locus are selected from the group consisting of Sobic.002G023300, Sobic.002G023500, Sobic.002G023600. In some cases, the one or more genes in the IAP locus comprises Sobic.002G023300. In some cases, the one or more genes in the IAP locus comprises Sobic.002G023500. In some cases, wherein the one or more genes in the IAP locus comprises Sobic.002G023600. In some cases, the one or more genes in the IAP locus comprises Sobic.002G023300 and Sobic.002G023500. In some cases, the one or more genes in the IAP locus comprises Sobic.002G023300 and Sobic.002G023600. In some cases, the one or more genes in the IAP locus comprises Sobic.002G023500 and Sobic.002G023600. In some cases, the one or more genes in the IAP locus comprises Sobic.002G023300 and Sobic.002G023500 and Sobic.002G023600.

In some embodiments, the modification of the one or more genes in the IAP locus increases cross-compatibility between the genetically modified plant and other species of Sorghum. In some cases, the modification of the one or more genes in the IAP locus reduces expression of the one or more genes. In some cases, the modification of the one or more genes in the IAP locus results in one or more non-functional proteins encoded by the one or more genes.

In some embodiments, the modification of the one or more genes in the IAP locus decreases cross-compatibility between the genetically modified plant and other species of Sorghum. In some cases, the modification of the one or more genes in the IAP locus increases expression of the one or more genes.

In one aspect, the present disclosure provides a method of producing a genetically modified plant of the genus Sorghum, the method comprising introducing one or more heterologous nucleic acid molecules into a plant of the genus Sorghum in a manner to modify one or more genes contained in an inhibition of alien pollen (IAP) locus of the plant. In some cases, one or more heterologous nucleic acid molecules are introduced into the plant of the genus Sorghum in a CRISPR/Cas9 system comprising guide RNAs directed to the one or more genes. In some embodiments, the genetically modified plant is of the species Sorghum bicolor.

In some embodiments of the method, the one or more genes in the IAP locus are selected from the group consisting of Sobic.002G023300, Sobic.002G023400, Sobic.002G023500, and Sobic.002G023600, and Sobic.002G023700. In some cases, the one or more genes in the IAP locus are selected from the group consisting of Sobic.002G023300, Sobic.002G023500, Sobic.002G023600. In some cases, the one or more genes in the IAP locus comprises Sobic.002G023300. In some cases, the one or more genes in the IAP locus comprises Sobic.002G023500. In some cases, the one or more genes in the IAP locus comprises Sobic.002G023600. In some cases, the one or more genes in the IAP locus comprises Sobic.002G023300 and Sobic.002G023500. In some cases, the one or more genes in the IAP locus comprises Sobic.002G023300 and Sobic.002G023600. In some cases, the one or more genes in the IAP locus comprises Sobic.002G023500 and Sobic.002G023600. In some cases, the one or more genes in the IAP locus comprises Sobic.002G023300 and Sobic.002G023500 and Sobic.002G023600.

In some embodiments of the method, the guide RNAs are selected from the group consisting of SEQ ID NOs: 8-11, 13-16, and 18-21.

In some embodiments, introduction of the one or more heterologous nucleic acid molecules knocks out functional expression of the one or more genes of the IAP locus.

In one aspect, the present disclosure provides a genetically modified plant of genus Sorghum produced by the method discussed above or herein.

In one aspect, the present disclosure provides a method of producing progeny of a genetically modified plant of the genus Sorghum, wherein the method comprises cultivating a genetically modified plant produced by the method discussed above to generate a population of progeny of the genetically modified plant.

In one aspect, the present disclosure provides a population of progeny of the genetically modified plant produced by the method discussed above or herein.

In one aspect, the present disclosure provides a method of producing hybrid progeny of a genetically modified plant of the genus Sorghum, wherein the method comprising cross-pollinating a genetically modified plant produced by the method discussed above with one or more other species of Sorghum to generate a genetically modified hybrid progeny, and cultivating the genetically modified hybrid progeny to generate a population of hybrid progeny of the genetically modified hybrid progeny. In some embodiments, the hybrid progeny exhibits greater heat tolerance than the plant of the genus Sorghum. In some embodiments, the hybrid progeny exhibits greater drought tolerance than the plant of the genus Sorghum.

In one aspect, the present disclosure provides a population of hybrid progeny of the genetically modified hybrid progeny produced by the method discussed above or herein.

In one aspect, the present disclosure provides a method of increasing cross-compatibility of a first plant of the genus Sorghum with a second plant of one or more other species of the genus Sorghum, wherein the method comprises genetically modifying the first plant by modifying one or more genes contained in an inhibition of alien pollen (IAP) locus of the first plant to produce a genetically modified plant, wherein the one or more genes controls cross-incompatibility of the first plant with other species of Sorghum and the modification of the one or more genes of the IAP locus reduces expression of the one or more genes, or results in one or more non-functional proteins encoded by the one or more genes.

In one aspect, the present disclosure provides a method of decreasing cross-compatibility of a first plant of the genus Sorghum with a second plant of one or more other species of the genus Sorghum, wherein the method comprises genetically modifying the first plant by modifying one or more genes contained in an inhibition of alien pollen (IAP) locus of the first plant to produce a genetically modified plant, wherein the one or more genes controls cross-incompatibility of the first plant with other species of Sorghum and the modification of the one or more genes of the IAP locus increases expression of the one or more genes.

In some embodiments of the two methods discussed immediately above, genetically modifying the first plant comprising introducing one or more heterologous nucleic acid molecules into the first plant in a CRISPR/Cas9 system comprising guide RNAs directed to the one or more genes.

In some embodiments of the two methods discussed immediately above, the genetically modified plant is of the species Sorghum bicolor.

In some embodiments of the two methods discussed immediately above, the one or more genes in the IAP locus are selected from the group consisting of Sobic.002G023300, Sobic.002G023400, Sobic.002G023500, and Sobic.002G023600, and Sobic.002G023700. In some cases, the one or more genes in the IAP locus are selected from the group consisting of Sobic.002G023300, Sobic.002G023500, Sobic.002G023600. In some cases, the one or more genes in the IAP locus comprises Sobic.002G023300. In some cases, the one or more genes in the IAP locus comprises Sobic.002G023500. In some cases, the one or more genes in the IAP locus comprises Sobic.002G023600. In some cases, the one or more genes in the IAP locus comprises Sobic.002G023300 and Sobic.002G023500. In some cases, the one or more genes in the IAP locus comprises Sobic.002G023300 and Sobic.002G023600. In some cases, the one or more genes in the IAP locus comprises Sobic.002G023500 and Sobic.002G023600. In some cases, the one or more genes in the IAP locus comprises Sobic.002G023300 and Sobic.002G023500 and Sobic.002G023600.

In some embodiments of the two methods discussed immediately above, the guide RNAs are selected from the group consisting of SEQ ID NOs: 8-11, 13-16, and 18-21.

Other embodiments will become apparent from a review of the ensuing detailed description.

### BRIEF DESCRIPTION OF THE DRAWINGS

FIG. 1 illustrates synteny between Chromosome 5 in Sorghum bicolor and the Zea mays reference genome, including the Ga1 locus on Chromosome 2 and additional sections of Chromosome 4. Sorghum appears to retain the ancestral Poaceae locus that was divided following an ancient tetraploidization event in Zea. Homologous regions are shown with connecting strands.

FIG. 2 illustrates pollen germination of Sorghum plumosum and Sorghum bicolor in cross-pollination assays of Example 5. As shown, S. plumosum pollen germinated (A) and grew (B) in wild-type S. bicolor pistils but growth was arrested after 24 hours at the boundary between the stigma and style (C). Conversely, wild-type S. bicolor pollen in wild-type S. bicolor pistils germinated (D), grew (E), and reached the ovary (F) within 24 hours.

FIG. 3 shows the expression levels of candidate genes in the IAP locus in various sorghum flower tissue types as described in Example 5.

FIG. 4 shows an agarose gel (0.4%) as prepared in Example 4 with Johnsongrass Amplicons for IAP and BAM1, putative cross-incompatibility loci. Chr02:2144633 . . . 2160696: IAP full-length amplicon containing five candidate genes; Chr02:2144633 . . . 2150496: sub-region of IAP containing two candidate genes expressed in floral tissues at anthesis, Sobic.002G023300.1 and Sobic.002G023400; Chr02:2550778 . . . 2556242: amplicon containing the full-length BAM1 gene (Sobic.002G027600.1).

FIG. 5 illustrates a heatmap of the Johnsongrass de novo genome assembly. The boxes diagonally across the center denote scaffolds and contigs. The diagonal lines on either side of the center are the result of synteny between those contigs. The gap in the diagonal lines appears to be one chromosome that is complicated. There is more polyploidy, heterozygosity, or both than the other chromosomes. It is likely that this unresolved heterozygosity is the reason for only 36 chromosomes (scaffolds) being detected despite Johnsongrass having 40 chromosomes.

FIG. 6 illustrates ordering metrics for the Johnsongrass de novo genome assembly. A total of 36 finished scaffolds (chromosomes) were detected of N50=43,822,357 bp each.

FIG. 7 illustrates a phylogenetic tree of BAM1. The neighbor-joining method and distance corrections were conducted in MUSCLE of the coding region of the gene BAM1 in S. bicolor (designated Sorghum) against the Poaceae species Maize, Rice, Johnsongrass, and outside group Arabidopsis.

FIG. 8 illustrates a phylogenetic tree of Sobic.002G023300. The neighbor-joining method and distance corrections were conducted in MUSCLE of the coding region of the gene Sobic.002G023300 in S. bicolor (designated Sorghum) against the Poaceae species Maize, Rice, Johnsongrass, and outside group Arabidopsis.

FIG. 9 illustrates sorghum plants successfully transformed with the CRISPR constructs disclosed herein expressing the reporter gene tdTomato-ER when leaves are under fluorescence in Example 8. The fluorescing tdTomato-ER is seen as light grey.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention. Any specific value discussed above or herein may be combined with another related value discussed above or herein to recite a range with the values representing the upper and lower ends of the range, and such ranges are encompassed within the scope of the present disclosure.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Selected Abbreviations

IAP: Inhibition of Alien Pollen
GE: Genetically Edited or Genetically Engineered
GMO: Genetically Modified Organism
CC: Cross-compatibility
CI: Cross-incompatibility
CRISPR: Clustered Regularly Interspaced Short Palindromic Repeat (a region in bacterial genomes used in pathogen defense)
RNA: Ribonucleic acid
g DNA: Genomic deoxyribonucleic acid
crRNA: Endogenous bacterial RNA conferring target specificity (requires tracrRNA to bind to Cas9)
tracrRNA: Trans-activating CRISPR RNA
gRNA: guide RNA (a fusion of crRNA and tracrRNA)
gRNA sequence: 20 nucleotides preceding a PAM sequence in genomic DNA
PAM: Protospacer Adjacent Motif
CDS: Coding Sequence
UTR: Untranslated region
PR1-LIKE: Pathogenesis-Related 1-like
CRISPs: Cysteine-rich secretory proteins
HMW: High molecular weight
RNase A: Ribonuclease A
PFGE: Pulsed-field gel electrophoresis
SNPs: Single nucleotide polymorphisms
RNA-Seq: RNA sequencing
PCR: Polymerase Chain Reaction
InDel: Insertion/Deletion
bp: Base Pair
kb: Kilobase
ORF: Open Reading Frame
NHEJ: Non-Homologous End-Joining
HDR: Homology Directed Repair
DSB: Double Strand Break

### Definitions

CRISPR Genome Editing or Engineering: The terms “CRISPR” or “CRISPR Genome Editing” or “CRISPR Genome Engineering” as used herein refer to the Clustered Regularly Interspaced Short Palindromic Repeats (CRISPR) Type II system, which is the most commonly used RNA-Guided Endonuclease technology for genome engineering. There are two components to this system: (1) a guide RNA and (2) an endonuclease, in this case the CRISPR associated (Cas) nuclease, Cas9. Guide RNA is a combination of the targeting specificity of endogenous bacterial crRNA and the scaffolding properties of tracrRNA into a single chimeric guide RNA (gRNA) transcript. When the gRNA and the Cas9 are expressed in a cell, the genomic target sequence can be modified or permanently disrupted.

The gRNA/Cas9 complex is recruited to a target sequence by the base-pairing between the gRNA sequence and the complement to the target sequence in the genomic DNA. For successful activity of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the wild-type Cas9 can cut both strands of the DNA causing a Double Strand Break (DSB).

A DSB can be repaired through one of two repair pathways: (1) the Non-Homologous End Joining (NHEJ) DNA repair pathway or (2) the Homology Directed Repair (HDR) pathway. The NHEJ repair pathway typically results in nucleotide inserts/deletions (InDels) or substitution at the DSB site that can lead to, for example, frameshifts and/or premature stop codons, effectively disrupting the open reading frame (ORF) of the targeted gene. The HDR pathway requires the presence of a repair nucleic acid template, which is used to fix the DSB. HDR faithfully copies the sequence of the repair template to the cut target sequence. Specific nucleotide changes can be introduced into a targeted gene by the use of HDR with a repair template carrying the desired change.

Cas9 nuclease: The terms “Cas9 nuclease” or “Cas9” as used herein refer to an endonuclease having two functional domains: RuvC and HNH, each cutting a different DNA strand. When both of these domains are active as in wild-type Cas9, the Cas9 causes double strand breaks (DSBs) in the genomic DNA. In the absence of a suitable repair template, the DSB is then repaired by the Non-Homologous End Joining (NHEJ) DNA repair pathway. During NHEJ repair, InDels (insertions/deletions) or substitutions can occur as a small number of nucleotides as either inserted or deleted at random at the DSB site, thereby altering the Open Reading Frame (ORF) of the target gene, which may significantly change the amino acid sequence downstream of the DSB. Additionally, InDels can also introduce a premature stop codon either by creating one at the DSB or by shifting the reading frame to create one downstream of the DSB. However, InDels induced by NHEJ are random, so the type and extent of gene disruption will need to be determined experimentally. To maximize the effect of gene disruption, target sequences preferably are chosen near the N-terminus of the coding region of the gene of interest, typically to introduce a DSB within the first or second exon of the gene.

The CRISPR system can also be used to introduce specific nucleotide modifications of the target sequence. Thus, cells can utilize a less error-prone DNA repair mechanism termed “Homology Directed Repair (HDR).” To introduce nucleotide modifications to genomic DNA, a DNA repair template containing the desired sequence must be provided during HDR. The DNA template is typically transfected into a cell together with the gRNA/Cas9 and must have a high degree of complementarity to the nucleotide sequences immediately upstream and downstream of the DSB. The length and binding position of each “homology arm” is dependent on the size of the change being introduced. When designing a repair template for genome editing by HDR, the repair template must not contain the target sequence followed by the PAM sequence or the template itself will also be cut by the Cas9. Changing the sequence of the PAM in the repair template likely will prove sufficient to ensure it is not cut by Cas9.

Close Relatives: The term “close relatives” refers to plant relatives of S. bicolor which are naturally cross-compatible with wildtype S. bicolor. For example, close relatives include Sorghum halepense (Johnsongrass).

Distant Relatives: The term “distant relatives” refers to plant relatives of S. bicolor which are cross-incompatible with wild type S. bicolor. For example, Saccharum officinarum L. (sugarcane), and Sorghum plumosum (wild Australian Sorghum relative) are distant relatives of S. bicolor and cross-incompatible with wildtype S. bicolor.

Off-Target Effects: The term “Off-Target Effects” as used herein refers to when flexibility in the base-pairing interactions between the gRNA sequence and the genomic DNA target sequence allows imperfect matches to the target sequence to be cut by Cas9. Single mismatches at the 5′ end of the gRNA (furthest from the PAM site) can be permissive for off-target cleavage by Cas9.

Nickase: The term “nickase” as used herein refers to modified versions of the Cas9 enzyme containing a single inactive catalytic domain, either RuvC- or HNH—. With only one active nuclease domain, the Cas9 “nickase” will cut only one strand of the target DNA, thereby generating a single-strand break or ‘nick’. A Cas9 nickase is still able to bind DNA based on gRNA specificity, though nickases will only cut one of the DNA strands. The majority of CRISPR plasmids are derived from S. pyogenes and the RuvC domain inactivated, for example, by a D10A mutation.

A single-strand break, or nick, is normally repaired through the HDR pathway, using the intact complementary DNA strand as the template. However, two proximal, opposite strand nicks introduced by a Cas9 nickase are treated as a Double Strand Break (DSB), in what is often referred to as a ‘double nick’ or ‘dual nickase’ CRISPR system. A double-nick induced DSB can be repaired by either NHEJ or HDR depending on the desired effect on the gene target. For example, two different gRNAs can bind in a particular genomic region. When the gRNAs are co-expressed with a Cas9 nickase, single-strand nicks created in the DNA are quickly repaired by HDR using the intact compliment strand as a template and no change occurs. Nicks in close proximity (and on opposite strands) behave as a DSB. However, by situating two gRNAs with target sequences in close proximity and on opposite strands of the genomic DNA, off-target effects of either gRNA alone will result in nicks that will not change the genomic DNA. Only at the target location where both nicks are proximal, will the double nicked sequence be considered a DSB. The double-nickase technique is described in, for example, Ran et al., (2013) Cell 154: 1380-1389.

The Cas9 protein requires the targeting specificity of a gRNA. Choosing an appropriate target sequence in the genomic DNA is a crucial step in designing an experiment. The target sequence is 20 nucleotides followed by the appropriate Protospacer Adjacent Motif (PAM) sequence in the genomic DNA. Target sequences (20 nucleotides+PAM) can be on either strand of the genomic DNA and can appear in multiple places in the genome. Accordingly, a bioinformatic program is helpful to select target sequences and minimize off-target effects. There are a number of tools available to help choose/design target sequences as well as lists of bioinformatically determined (but not experimentally validated) unique gRNAs for different genes in different species.

Protospacer Adjacent Motif (PAM) Sequence: The term “Protospacer Adjacent Motif (PAM) Sequence” as used herein refers to a nucleic acid sequence present in the DNA target sequence but not in the gRNA sequence itself. For Cas9 to successfully bind to DNA, the target sequence in the genomic DNA must be complementary to the gRNA sequence and must be immediately followed by the correct protospacer adjacent motif or PAM sequence. Any DNA sequence with the correct target sequence followed by the PAM sequence will be bound by Cas9. A target sequence without the PAM following it is not sufficient for Cas9 to cut. Furthermore, the PAM sequence varies by the species of the bacteria from which the Cas9 was derived. The Type II CRISPR system derived from S. pyogenes, for example, has the PAM sequence NGG located on the immediate 3′ end of a gRNA recognition sequence and components (gRNA, Cas9) derived from different bacteria will not function together. The CRISPR system requires that both the gRNA and Cas9 are expressed in the target cells, the respective promoters for Cas9 and gRNA expression determining the species specificity of a particular system.

Whichever sequences and hybridization methods are used, one skilled in the art can readily determine suitable hybridization conditions, such as temperature and chemical conditions. Such hybridization methods are well known in the art. For example, for applications requiring high selectivity, one will typically desire to employ relatively stringent conditions for the hybridization reactions, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand, and are particularly suitable for detecting specific SNPs according to the present invention. It is generally appreciated that conditions can be rendered more stringent by the addition of increasing amounts of formamide. Other variations in hybridization reaction conditions are well known in the art (see for example, Sambrook et al., Molecular Cloning; A Laboratory Manual 2d ed. (1989)).

Polymerase Chain Reaction or PCR: The terms “polymerase chain reaction” or “PCR” as used herein refer to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application. PCR may generate thousands to millions of copies of a particular DNA or RNA sequence being amplified. PCR is commonly used to amplify the number of copies of a DNA or RNA segment for cloning or to be used in other analytical procedures.

Primer: The term “primer” as used herein refers to an oligonucleotide, the sequence of at least a portion of which is complementary to a segment of a template DNA which is to be amplified or replicated. Typically, primers are used in performing the polymerase chain reaction (PCR). A primer hybridizes with (or “anneals” to) the template DNA and is used by the polymerase enzyme as the starting point for the replication/amplification process. By “complementary” it is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

Amplification: As used herein, “amplification” refers to the production of multiple copies of a segment of DNA or RNA. Amplification is usually induced by polymerase chain reaction.

Nucleic acid: The term “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA), or modified or substituted sugar or phosphate groups.

Heterologous: The terms “heterologous” or “exogenous” mean derived from a genotypically distinct entity from that of the rest of the entity to which it is compared or into which it is introduced or incorporated. For example, a nucleic acid introduced by genetic engineering techniques into a different cell type is a heterologous nucleic acid (and, when expressed, can encode a heterologous polypeptide). Similarly, a cellular sequence (e.g., a gene or portion thereof) that is incorporated into a plant cell is a heterologous or exogenous nucleotide sequence with respect to the plant cell.

Vector: A “vector,” as used herein, refers to a recombinant plasmid that comprises a nucleic acid to be delivered into a host cell, either in vitro or in vivo. A vector can comprise DNA or RNA, for example.

Read: The term “read” with regard to sequencing refers to the nucleic acid sequence of a cluster of nucleotides that is obtained after the end of the sequencing process and which is ultimately the sequence of a section of a complete nucleic acid sequence. A “read” is the base called value of a string of nucleotides derived from a raw signal.

Long-read sequencing: The term “long-read sequencing” refers to a DNA sequencing technique which can determine a sequence of nucleotides of long sequences of DNA between 10,000 and 100,000 base pairs at a time, thereby eliminating the need to fragment and amplify DNA normally required in other DNA sequencing techniques.

High molecular weight (HMW) DNA: The term “HMW DNA,” as used herein, refers to a DNA larger than 50 kb. HMW DNA is generally useful for long-read next-generation sequencing and studies that investigate large-scale genomic variation such as structural variation. HMW DNA is also crucial in functional metagenomic studies, because large fragments present greater access to genes of interest.

Linked Reads: The term “linked reads,” as used herein, refers to a sequencing technology that leverages microfluidics to partition and barcode HMW DNA to generate a data type that provides contextual information of the genome from short-reads. The use of linked read sequencing data may be extended for the purpose of resolving complex genomic structural rearrangements.

Contig: The term “contig,” as used herein, refers to a set of DNA segments or sequences that overlap in a way that provides a contiguous representation of a genomic region.

Polyploid: The term “polyploid,” as used herein, refers to species in which three or more sets of chromosomes coexist. Polyploidy frequently occurs in plants and plays a major role in their evolution. Based on their origin, polyploid species can be divided into two groups: autopolyploids and allopolyploids. The autopolyploids arise by multiplication of the chromosome sets from a single species, whereas allopolyploids emerge from the hybridization between distinct species followed or preceded by whole genome duplication, leading to the combination of divergent genomes. Polyploidization is a key mechanism of speciation in the grass family Poaceae.

“Genetically Edited” or “Genetically Engineered” or “Genetically Modified”: Any genetic change in the genome of an organism induced by molecular mechanisms, for example, by CRISPR/Cas9. The genetic change can be a nucleotide insertion, deletion, or substitution in a target gene or genome location to yield a genotypic or phenotypic change in the edited organism. The target gene or genome location can be, for example, a protein coding gene, a promoter sequence, a cis or trans regulatory element, a transcription factor gene, a transposon, or a non-coding sequence. In a protein coding gene, the nucleotide insertion, deletion, or substitution can lead to a premature stop codon, the loss of a start codon, and/or a mutation that results in a loss or change of function in the RNA or protein associated with the transcription or translation of the edited gene containing the insertion, deletion, or substitution. An insertion can be the addition of 1, 2, 3, 4, 5, 6, 7, 8, 9, 10 or more nucleotides in length to the genome location. A deletion can be the removal of 1, 2, 3, 4, 5, 6, 7, 8, 9, 10 or more nucleotides in length from the genome location. Genetically editing, engineering or modifying can also affect the genetic expression of a target gene by either increasing, decreasing, or eliminating expression of said gene.

### Overview

The present disclosure reveals the identification of key genes in the IAP locus of S. bicolor that control cross-compatibility. These genes include Sobic.002G023300, Sobic.002G023400 Sobic.002G023500, Sobic.002G023600, and Sobic.002G023700.

Sobic.002G023300 encodes a cysteine-rich secretory defense-related protein that is highly expressed in floral tissues at anthesis. Cysteine-rich secretory proteins (CRISPs) play a role in host-recognition signaling and are involved in both the activation of defense-related mechanisms and pre-zygotic reproductive barriers (Takayama, et al., 2001, Nature 413, 534-538; Wheeler, et al., 2009, Nature 459, 992-995). One such CRISP in Arabidopsis, Wak1 (Decreux & Messiaen, 2005, Plant and Cell Physiology 46(2), 268-278), has a role in both mechanisms by functioning as a potential sensor of cell wall signaling by directly binding to the calcium that is cross-linked when PMEs de-methylate homogalacturonan.

The present disclosure demonstrates the use of CRISPR/Cas9 genome engineering to create mutant Sorghum bicolor lines in five genes identified at the IAP locus. The individual mutations created by the CRISPR/Cas9 system in cultivated sorghum (Sorghum bicolor), according to the present disclosure, are directed to knockout the functions of the genes. Table 1 summarizes all the knock-out mutant lines that were created using the CRISPR/Cas9 system. More details regarding the CRISPR/Cas9 constructs and gRNA selection for each mutant line are provided below.

TABLE 1
Genes fine-mapped to IAP using the current version
of the S. bicolor reference genome (v3.1.1)
and production of CRISPR/Cas9 mutant lines

| | | Expressed in | Expressed in |
|---|---|---|---|
| | Alternative | S. bicolor | S. halepense |
| IAP gene | Name | pollen, pistils | pollen, pistils |
| Sobic.002G023300 | SbPR1-LIKE | pistils, pollen | pistils and pollen |
| Sobic.002G023400 | IAP2 | N/A | N/A |
| Sobic.002G023500 | IAP3 | pistils, pollen | pistils; highly |
| | | | specific |
| Sobic.002G023600 | IAP4 | pistils, pollen | pistils, pollen |
| Sobic.002G023700 | IAP5 | N/A | N/A |

### EXAMPLES

Additional details regards the experiments discussed above are provided in the following paragraphs. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1—Assessing Whether Genes in the IAP Locus are Homologous to Known Cross-Incompatibility Systems

Genes in the IAP locus do not share homology with genes in the maize Ga1 locus, a known cross-incompatibility system.

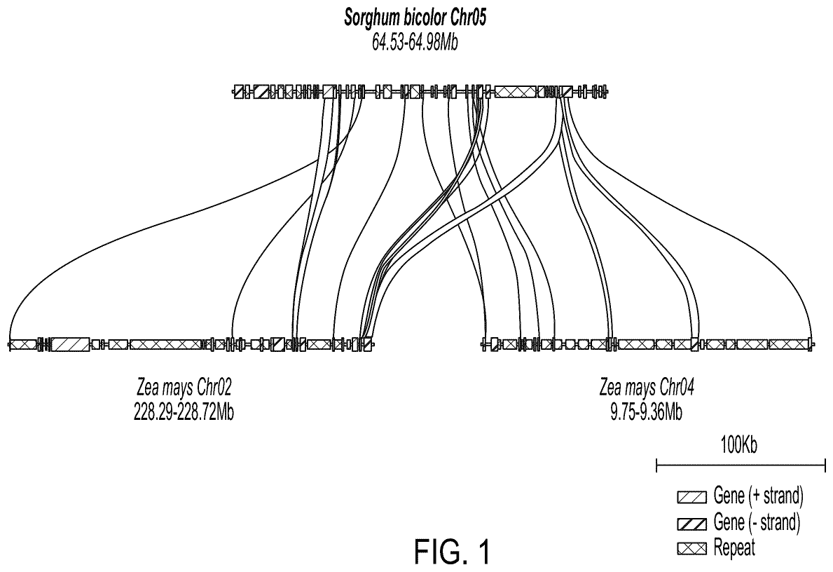

To assess if IAP is homologous to maize Ga1, a comparative genomics was completed. FIG. 1 illustrates synteny between Chromosome 5 in Sorghum bicolor and the Zea mays reference genome, including the Ga1 (Gametophyte 1) locus on Chromosome 2 and additional sections of Chromosome 4. While synteny exists between maize and S. bicolor reference genomes at the maize Ga1 locus, genes within the locus do not appear to have the same function in S. bicolor as in maize. This is due to the distribution of the ancestral Poaceae Ga1 locus among two chromosomes in Zea mays, which provides evidence that its evolution as a reproductive barrier followed the divergence of the two species' genomes ˜11 million years ago and after a whole-genome duplication event in Zea mays ˜4 million years ago which may have enabled the evolution of new functions. Moreover, no candidate genes at this locus in S. bicolor are pectin methylesterases (PMEs) or pectin methylesterase inhibitors (PME1s); and while there is a PMEI elsewhere on this chromosome, it is not expressed during anthesis. Therefore, maize Ga1 is not homologous to sorghum IAP.

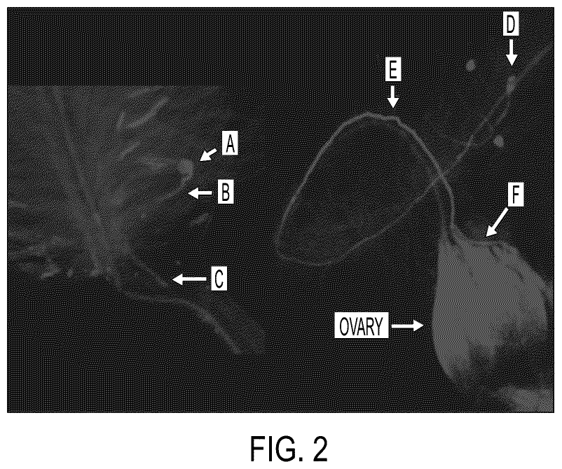

Sorghum bicolor is known to have an unrelated (based on synteny analysis) prezygotic reproductive barrier locus from known Zea loci at IAP (Inhibition of Alien Pollen) locus. The IAP locus controls two distinct phenotypes and, when homozygous recessive, allows for intra- and extrageneric pollen to grow normally to the ovary. In cases where pollen tubes reach the ovary, ploidy appears to be the primary determinate of whether fertilization occurs. A cross-incompatibility system has been physically observed between cultivated S. bicolor pistils and wild Sorghum species pollen in the crop's tertiary gene pool, mediated by the action of a pistil-expressed pollen killer at the wild-type IAP locus. Maize and wild Sorghum spp. pollen placed on wild-type IAP S. bicolor pistils showed stunted germination and inhibited growth patterns (e.g., as shown in FIG. 2), similar to that observed during incompatible pollen-pistil interactions conferred by Ga1, Gat, and Tcb1 loci in maize, rarely (<0.1%) reaching the sorghum ovary. Pollen from both maize and wild Sorghum species (including Johnsongrass) displays a range of genotype-specific compatibility and incompatibility with S. bicolor pistils (Hodnett, et al., 2005, Crop Science 45, 1403-1409), which is overcome (i.e., cross-compatibility is restored) when sorghum pistils carry homozygous recessive (iap iap) alleles.

### Example 2—Identification of Candidate Genes at the S. bicolor IAP Locus

The S. bicolor IAP locus was fine mapped to identify specific genes present in locus. The sequences were BLASTed into the current version (v3.1.1) of the sorghum genome, which was assembled using long reads and is therefore a more reliable assembly, revealed that there are in fact five tightly linked candidate genes at IAP (Sobic.002G023300 through Sobic.002G023700) in v3.1.1. Three of these genes (Sobic.002G023300, Sobic.002G023500, and Sobic.002G023600) are predicted by Gramene (data release 63) and Phytozome (v12.1.5) to be expressed in floral tissues (inflorescences, pollen, and/or pistils) at anthesis. Moreover, the pistil-expressed genes are candidates because a system for dealing with incompatible pollen would need to be constitutively expressed in mature pistils in order to detect and/or inhibit it.

As disclosed, the investigations of candidate genes at IAP locus identified, out of the three genes, one gene (Sobic.002G023300) encoding a cysteine-rich secretory defense-related protein that is highly expressed in floral tissues at anthesis. Cysteine-rich secretory proteins (CRISPs) play a role in host-recognition signaling and are involved in both the activation of defense-related mechanisms and pre-zygotic reproductive barriers. Sobic.002G023300 is predicted to encode Pathogenesis-Related 1-like CRISPs, which are commonly expressed in pollen and pistils. As described above, this gene also seems to be the most well conserved across the IAP and BAM1 loci between Sorghum and closely related Poaceae crops. Sobic.002G023500 is included on both Gramene and Phytozome, and is highly expressed in pistils, but it also has medium expression in pollen. Interestingly, Sobic.002G023500 was highly expressed and highly specific to pistils in the pistil library of RNA-Seq study that was used to generate annotations for a de novo Johnsongrass genome assembly.

### Example 3— Bioinformatics Analysis of Johnsongrass IAP and BAM1

Sorghum halepense (Johnsongrass) is a noxious weed in 46 states in the United States and often found growing within close proximity to S. bicolor, where it has been shown to contaminate harvested seed through pollen-mediated gene flow. The risk of gene flow from S. bicolor to Johnsongrass is the primary reason why genetically engineered S. bicolor has not been approved for commercialization. Current genetic evidence indicates that Johnsongrass (2n=4x=40) is an allopolyploid descendent of S. bicolor (2n=2x=20) and S. propinquum (2n=2x=20) (Paterson, 2009, Nature 456-551), hence the observed cross-compatibility between feral S. bicolor and Johnsongrass (Ohadi, et al., 2017, Critical Reviews in Plant Sciences 36:5-6, 367-385). Therefore, genetic studies on the IAP locus in Johnsongrass can shed light on the corresponding genes and their functions in the S. bicolor IAP locus.

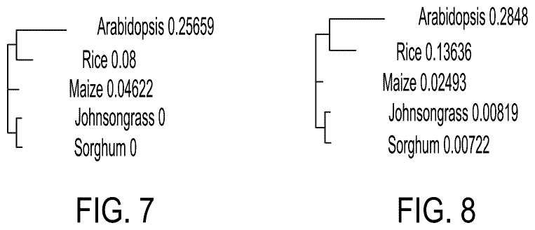

Multiple sequence alignment for the coding regions of each candidate gene within the amplicons containing the full-length Johnsongrass IAP region and BAM1 was performed using ClustalW in MUSCLE (About UniProt., 2019). The consensus DNA sequence of each Johnsongrass amplicon sequenced in PacBio containing putative genes associated with the BAM1 and IAP loci in sorghum was BLASTed in Phytotozome against the Sorghum bicolor v3.3.1, Oryza sativa v7_JGI (rice), Zea mays PH207 v1.1 (maize), and Arabidopsis thaliana TAIR10 reference genomes. Any genes in reverse orientation to the S. bicolor reference sequence were reverse complimented prior to running in MUSCLE. Phylogenetic trees were constructed based on sequence alignments at these loci. The alignment between the S. bicolor reference genome and the full-length IAP amplicon of Johnsongrass showed 97.3% identity, whereas the alignment of the BAM1 amplicon showed 100% identity. The phylogenetic tree of the coding region of Johnsongrass BAM1 (FIGS. 7 and 8) reflects the expected evolutionary relationships, with S. bicolor and Johnsongrass being very closely related, followed by maize, rice, and Arabidopsis, respectively.

Alignments using putative coding regions of the five candidate genes in the full-length IAP amplicon of Johnsongrass clearly showed Sobic.002G023300.1 as the most conserved sequence across genera. Interestingly, the other genes at IAP had no strong hits in the other Poaceae genomes (rice and maize), and were absent from Arabidopsis, indicating the unique nature of this locus to Sorghum. The Johnsongrass homologue of Sobic.002G023300.1 also reflected the expected evolutionary relationships described for BAM1.

These results indicate that both sequence divergence and structural rearrangements have occurred within this region during its evolution as a putative prezygotic reproductive barrier loci unique to Sorghum, similar to the structural rearrangements detected within each of the Ga1, Gat, and Tcb1 maize loci and the remaining Zea species. While structural rearrangements underpinning reproductive barriers is not unexpected, probably the most unexpected result of this work is that no known prezygotic reproductive barrier locus of Zea demonstrates any degree of synteny to IAP or BAM1 of S. bicolor or Johnsongrass, despite the close evolutionary relationship of these species. Moreover, while PME/PMEI systems appear conserved amongst all known maize prezygotic reproductive barrier loci, no PMEs or PMEIs were found to play a similar mechanistic role in these two Sorghum species.

### Example 4—Sequencing Putative Cross-Incompatibility Loci in the Johnsongrass Genome

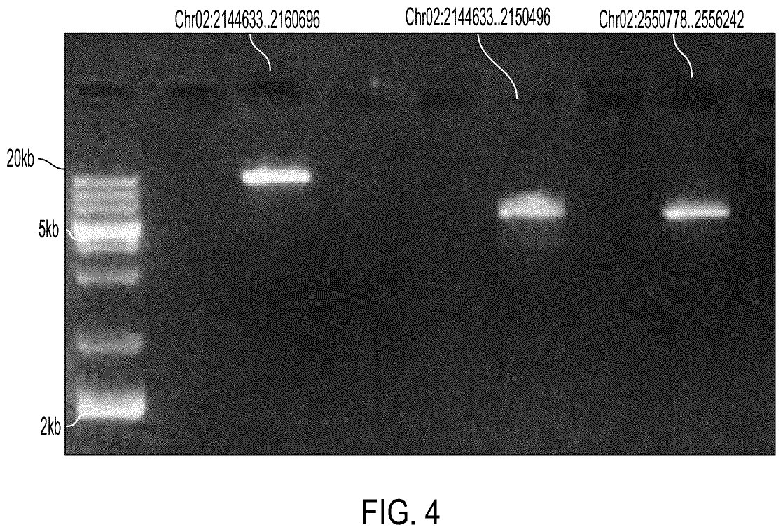

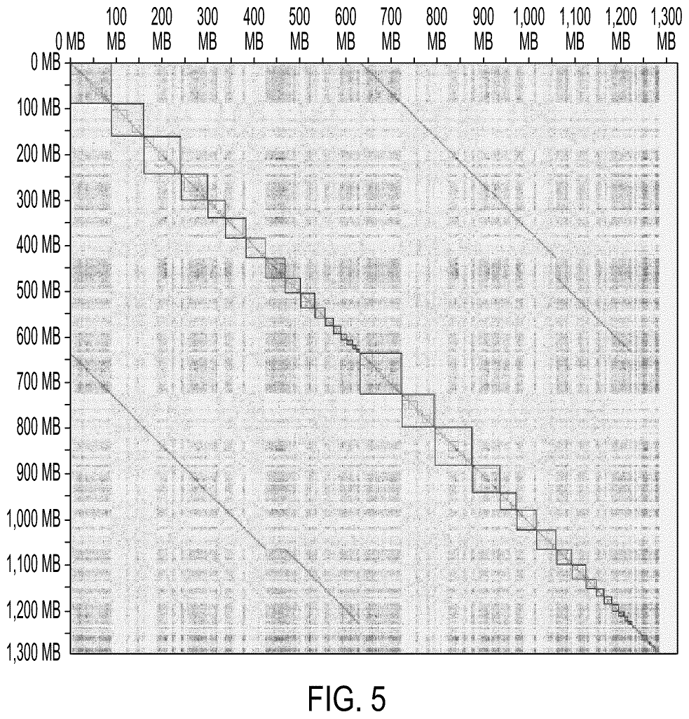

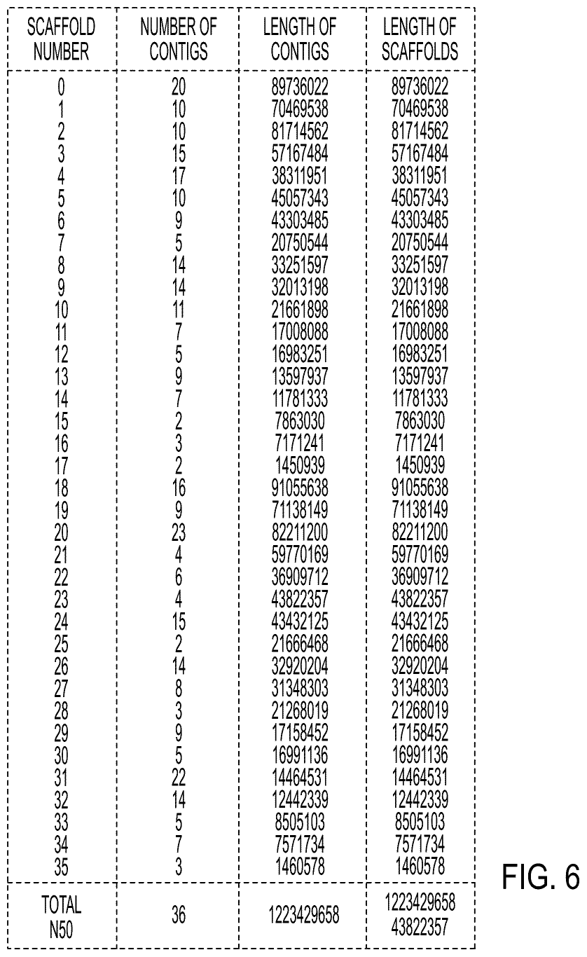

Based on mapping, the candidate cross-incapability region in Johnsongrass was actually distributed among two distinct regions of Chromosome 2, spaced about 40 kb apart in. The first section is referred to as IAP (containing five candidate genes) and the second section is referred to as BAM1 (containing only one candidate gene), based on nucleotide sequence homology among the two versions of the reference genome. The re-sequencing of putative reproductive barrier loci and candidate genes were completed by two different techniques. The full IAP locus (roughly 16 kb) was amplified in a two-step PCR cycle using Takara LA Taq DNA polymerase (Cat. #RR002A). The PCR reaction was prepared with the following components to measure a total of 50 μl: 500 ng of genomic high molecular weight Johnsongrass DNA, 5 μl of 10× LA PCR Buffer II with Mg²⁺, 8 μl of dNTPs, 0.5 μl of Takara LA Taq DNA polymerase, 32.86 μl of distilled water, 0.25 μl of the sense primer (5′-CGGTGACCATGCCAAGTACAGCAAATTAAC-3′) (SEQ ID NO: 1), and 0.25 μl of the antisense primer (5′-CGGCAGTGAGAATGTTTACTGTTTGCTCAT-3′) (SEQ ID NO: 2). The fragment was then amplified with a two-step PCR as follows: a denaturing cycle for 1 minute at 94° C. and 14 cycles alternating between 20 seconds at 98° C. and 20 minutes at 68° C.; followed by 16 cycles alternating between 20 seconds at 98° C. and 20 min+15 seconds/cycle at 68° C. The final extension was 10 min at 72° C. A 0.4% agarose gel was used to validate the length of the amplicon using 4 μl of the reaction (see the first marked well in FIG. 4. Chr02:2144633 . . . 2160696).

Takara PrimeSTAR® GXL Premix (Cat. #R051A) was used to amplify specific genes of interest within the IAP and BAM1 loci. 50 μl reactions were prepared with the following components: 500 ng of genomic HMW Johnsongrass DNA, 25 μl of PrimeSTAR GXL Premix (2x), 20.86 μl of distilled water and 0.25 μl of the sense and antisense primers for BAM1 (Forward: 5′-CAAAGAAA AGACAAGTTTCTCAAAAGATCA-3′ (SEQ ID NO: 3); Reverse: 5′-TCTGTATAACAAAGTAGTAGGAGTACTTGC-3′) (SEQ ID NO: 4) or IAP (Forward: 5′-CGGTGACCATGCCAAGTACAGCAAATTAAC-3′ (SEQ ID NO: 1); Reverse: 5′-ATGAGCAAAC AGTAAACATTCTCACTGCCG-3′) (SEQ ID NO: 5) respectively. A three-step PCR was used as follows: an initial 10 second denaturing step at 98° C., 30 cycles of 10 seconds at 98° C., 15 seconds at 60° C., and 6 minutes at 68° C., respectfully; and a final extension of 6 minutes at 68° C. An aliquot of 4 μl of the completed reactions were loaded onto a 0.4% agarose gel and used in electrophoresis to verify fragment lengths (see the second and third marked wells in FIG. 4).

### Example 5—Cross-Pollination Assays and Determining Expression Levels of Candidate Genes During a Compatible and Incompatible Cross

The expression of candidate IAP locus genes was assessed to better determine their potential role in controlling cross-incompatibility. Cross-pollination assays between “Tx623” sorghum (the source of the reference genome) and Sorghum plumosum, as Australian wild distant relative were conducted. Tx623 sorghum and S. plumosum are cross-incompatible.

During the cross-pollination assays, it was observed that the S. plumosum pollen tube was inhibited at the boundary between the stigma and style after 24 hours, whereas the pollen tube of the self-pollinated sample had reached the ovary by that time, as in seen in FIG. 2.

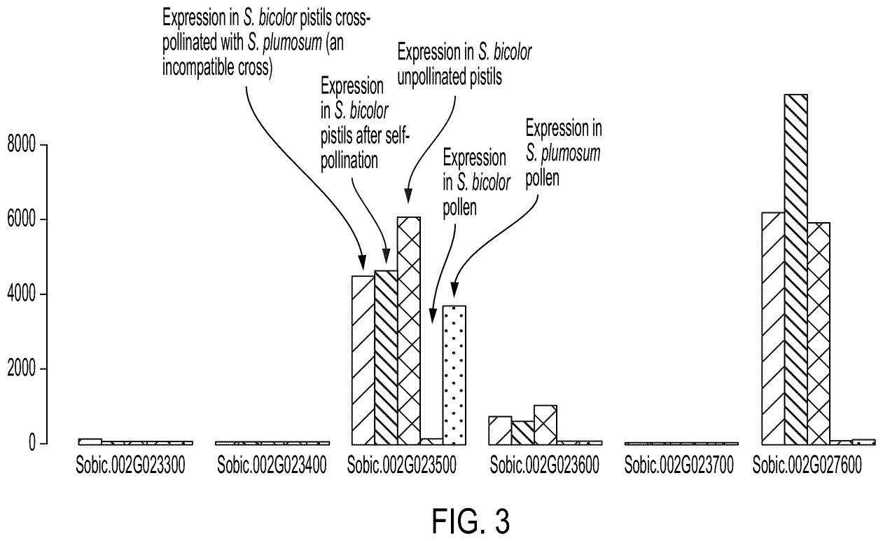

An RNA-seq study was conducted using the same plants, but at an earlier timepoint to try and capture the plant's initial signal responding to the detection of distant relative pollen. Samples submitted for RNA-Seq analysis included fresh pollen from sorghum and S. plumosum, unpollinated sorghum and S. plumosum pistils, self-pollinated sorghum and S. plumosum pistils, and sorghum pistils cross-pollinated with S. plumosum for 15-30 minutes. Results of the expression levels can be seen in FIG. 3. There is generally higher expression of IAP genes in pistils than in pollen, and (at least at the early timepoint evaluated) much higher expression of Sobic.002G023500 than of Sobic. 002g023300.

### Example 6—Production of Genetically Edited Sorghum Using CRISPR/Cas9 Gene Editing Tool

S. bicolor plants were transformed with CRISPR/Cas9 constructs. The CRISPR/Cas9 machinery is composed of Cas9 (a site-specific DNA endonuclease) and a synthetic single guide RNA (sgRNA, alternatively designated gRNA). The gRNA, which carries 20-nuclecotides of target sequence information, is used to direct the Cas9 endonuclease to its genomic target sequence, which must precede a tri-nucleotide sequence known as the protospacer-associated motif (PAM). Streptomyces pyogenes Cas9 recognizes the PAM sequence NGG and cleaves three nucleotides preceding this PAM sequence on complementary and non-complementary strands.

### Example 7—CRISPR/Cas9 Constructs and sRNA Selections

SEQ ID NO: 6 is the full-length Sorghum bicolor IAP region (Chr02:2144633 . . . 2160696). A PCR primer used to amplify long-reads in Johnsongrass is SEQ ID NO: 22.

CRISPR (http://crispor.tefor.net/) was used to search for guide RNA. The Sorghum bicolor genome (Phytozome V9, December 2012) and Sorghum bicolor— milo (NCBI GCF_000003195.3) were selected as the genetic background to determine the off-target potential of candidate guides. Current utilization of a soybean codon optimized version of the Streptococcus pyogenes Cas9 (SpCas9) is directed to cleave DNA in a sequence specific manner by a guide RNA of 20 nucleotides; these guides are located next to protospacer adjacent motifs (PAM) which are NGG.

Exemplary guide RNAs useful in a CRISPR/Cas9 system for editing target cross-incapability genes are shown in Table 2. Guide RNAs are selected based on specificity score, off-targets, and location. The guide RNAs are relative to the +1 (ATG) genomic DNA position.

TABLE 2
Guide RNAs Used for CRISPR/Cas9 System per Target IAP Gene

| Target Gene | Reverse Guide RNAs | Forward Guide RNAs |
|---|---|---|
| Sobic.002G023300 | gRR22 (SEQ ID NO: 8) | gRF179 (SEQ ID NO: 10) |
| (SEQ ID NO: 7) | gRR106 (SEQ ID NO: 9) | gRF255 (SEQ ID NO: 11) |
| Sobic.002G023500 | gRR62 (SEQ ID NO: 13) | gRF379 (SEQ ID NO: 15) |
| (SEQ ID NO: 12) | gRR1089 (SEQ ID NO: 14) | gRF588 (SEQ ID NO: 16) |
| Sobic.002G023600 | gRR1134 (SEQ ID NO: 18) | gRF42 (SEQ ID NO: 20) |
| (SEQ ID NO: 17) | gRR148 (SEQ ID NO: 19) | gRF275 (SEQ ID NO: 21) |

Sobic.002G02300 (SEQ ID NO: 7): Nucleotides 1−72=5′ UTR; Nucleotides 73-591=CDS; Nucleotides 592−739=3′ UTR

Sobic.002G023500 (SEQ ID NO: 12): Nucleotides 1−8=5′ UTR; Nucleotides 9-2459=CDS; Nucleotides 2460−2769=3′ UTR

Sobic.002G023600 (SEQ ID NO: 17): Nucleotides 1-167, 247-463, 1067-1189, 1367-1627, and 1725−1734=5′ UTR; Nucleotides 1735-2110, and 2237-2505=CDS; Nucleotides 2506-2654=3′ UTR

### Example 8—Confirmation of S. bicolor Transformants Stably Expressing Transgenes

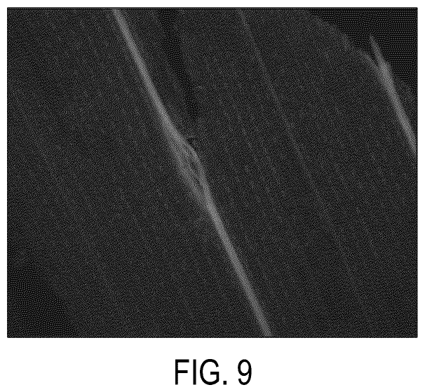

CRISPR/Cas constructs contained a tandem Tomato (tdTomato) reporter gene fused to an endoplasmic reticulum signal peptide. Leaf samples from the progeny of transformed S. bicolor parents were viewed under fluorescent wavelengths. Epifluorescent microscopy of the leaf samples was conducted using the tdTomato-ER filter set at 535/30 nm excitation and 600/50 nm band pass emission. Visual observations of the tdTomato fluorescing in transformants confirms the successful expression of transgenes. FIG. 9 shows the tdTomato-ER fluorescence in transformed sorghum leaf tissue.

1. A genetically modified plant of the genus species Sorghum bicolor, comprising a modification of one or more genes contained in an inhibition of alien pollen (IAP) locus of the plant, wherein the one or more genes controls cross-incompatibility of the plant with other species of Sorghum.

2. (canceled)

3. The genetically modified plant of claim 1, wherein the one or more genes in the IAP locus are selected from the group consisting of SbPR1-LIKE, IAP3, and IAP4.

4. (canceled)

5. The genetically modified plant of claim 3, wherein the one or more genes in the IAP locus comprises SbPR1-LIKE.

6. The genetically modified plant of claim 3, wherein the one or more genes in the IAP locus comprises IAP3.

7. The genetically modified plant of claim 3, wherein the one or more genes in the IAP locus comprises IAP4.

8.-10. (canceled)

11. The genetically modified plant of claim 1, wherein the one or more genes in the IAP locus comprises SbPR1-LIKE, IAP3, and IAP4.

12. The genetically modified plant of claim 11, wherein the modification of the one or more genes in the IAP locus increases cross-compatibility between the genetically modified plant and other species of Sorghum.

13. The genetically modified plant of claim 12, wherein the modification of the one or more genes in the IAP locus reduces expression of the one or more genes.

14. The genetically modified plant of claim 12, wherein the modification of the one or more genes in the IAP locus results in one or more non-functional proteins encoded by the one or more genes.

15.-16. (canceled)

17. A method of producing a genetically modified plant of the species Sorghum bicolor, the method comprising introducing one or more heterologous nucleic acid molecules into a plant of the species Sorghum bicolor in a manner to modify one or more genes contained in an inhibition of alien pollen (IAP) locus of the plant.

18. The method of claim 17, wherein the one or more heterologous nucleic acid molecules are introduced into the plant of the species Sorghum bicolor in a CRISPR/Cas9 system comprising guide RNAs directed to the one or more genes.

19. (canceled)

20. The method of claim 17, wherein the one or more genes in the TAP locus are selected from the group consisting of SbPR1-LIKE, IAP3, and IAP4.

21. (canceled)

22. The method of claim 20, wherein the one or more genes in the TAP locus comprises SbPR1-LIKE.

23. The method of claim 20, wherein the one or more genes in the TAP locus comprises IAP3.

24. The method of claim 20, wherein the one or more genes in the TAP locus comprises IAP4.

25.-29. (canceled)

30. The method of claim 17, wherein the introduction of the one or more heterologous nucleic acid molecules knocks out functional expression of the one or more genes of the TAP locus.

31. (canceled)

32. A method of producing a hybrid progeny of a genetically modified plant of the genus Sorghum, the method comprising cross-pollinating a genetically modified plant produced by the method of claim 17 with one or more other species of Sorghum to generate a genetically modified hybrid progeny, and cultivating the genetically modified hybrid progeny to generate a population of hybrid progeny of the genetically modified hybrid progeny.

33. The method of claim 32, wherein the hybrid progeny exhibits greater heat tolerance than the plant of the species Sorghum bicolor.

34. The method of claim 32, wherein the hybrid progeny exhibits greater drought tolerance than the plant of the species Sorghum bicolor.

35.-50. (canceled)

51. A population of hybrid progeny of the genetically modified hybrid progeny produced by the method of claim 32.


SEQUENCE LISTING (22 sequences)

SEQ ID NO: 1 - DNA, 30 bp, synthetic construct
  misc_feature 1..30: note = Synthetic
  source 1..30: mol_type = other DNA
cggtgaccatgccaagtacagcaaattaac

SEQ ID NO: 2 - DNA, 30 bp, synthetic construct
  misc_feature 1..30: note = Synthetic
  source 1..30: mol_type = other DNA
cggcagtgagaatgtttactgtttgctcat

SEQ ID NO: 3 - DNA, 30 bp, synthetic construct
  misc_feature 1..30: note = Synthetic
  source 1..30: mol_type = other DNA
caaagaaaagacaagtttctcaaaagatca

SEQ ID NO: 4 - DNA, 30 bp, synthetic construct
  misc_feature 1..30: note = Synthetic
  source 1..30: mol_type = other DNA
tctgtataacaaagtagtaggagtacttgc

SEQ ID NO: 5 - DNA, 30 bp, synthetic construct
  misc_feature 1..30: note = Synthetic
  source 1..30: mol_type = other DNA
atgagcaaacagtaaacattctcactgccg

SEQ ID NO: 6 - DNA, 16063 bp, synthetic construct
  misc_feature 1..16063: note = Synthetic
  source 1..16063: mol_type = other DNA
cattgaccctgacggtgaccatgccaagtacagcaaattaacagtcaccgtgttttactagtatatgccatcttgcttggacaagtctcactaaattcatcagtataaataccgtggccagtcgccacatctcatgcaccaaaatccatctaccacaacagcgcgccatctctgctcattcagataacttggtcagcagcaacagcggcaatggcgtcgtcctcctcgtcaccgacgaagctgctagcgtgcctcgtcgccgcagctctggcgctggctgccaccgtcgtcgcgccgtgcgcggcgcagaactcgccgcaggactacgtgaacccgcacaacgcggcgcgcgccgacgtcggcgtcggcccggtgtcgtgggacgacacggtggccgcgtacgcgcagagctacgcggcgcagcggcagggcgactgcaagctgatccactccggcggtccctacggcgagaacatcttctggggctccgccggcgccgactggtcggcgtccgacgccgtggcgtcgtgggtttccgagaagcagtactacaaccacgacaccaacagctgcgcggacggcaaggtgtgcgggcactacacgcaggtggtgtggcgtgactccacggccatcggctgcgcccgcgtcgtctgcgacaacaacgccggcgtcttcatcatctgcagctacaacccgccgggcaactacgtcggccagagcccatactagacgtagtagtgtgccgtatgcatgaattgaatacatgcaagtatacgtactggggtcggagtgaaaataaattgttgtcaactttataccatactatgaatgttgataaacataataagtcaataaaatcatgtgattgctgaggcatatatatttttgttcttatattttgatcgttaattactacagaaacgatctttaggaacatgcatgatcaattttagtactatactacaaccaatttgcagcgtcgcataacaagaccatgtgatttttcatatatgctattacatctgcccaaaaaaaataaaaataaacactactataaaacccattctcaatatcattttctccatctttacagtaaatgctactacgtccacctaaaaatgcacaataaacactttcacaatatcatttttttggcactgatgtagtaataacttattgtgataaatttgttatcactatcggttcgttacacaattgtggtaacatacaacaccacaatcggcccactagcctaatagaaaacactaaggctgtgtttggttctttggaatttggcccaggaacaattctatctcctcaagactatacaaattagtgaagcaatccaaaactaggaattattcgaggccctcattccagtagaaccgaacaaggcctaaggctttgtcctcactctatttcagactcttccctctctcatagccgctgctctagcaccgcctccacgtcccaagaccccaacgccaccgaactggagctctataatttcatcaatttttagttttaggtcccgtcaaactataaatttgatgtaattttttcatctgactttatttgaataaattataaattattcatgctatagctgtttttgaggtccatgcatgtttgaaatttgatttctaagtatggctgacttacctagaaatcattcattttagagtcggttgacataagcaaccatccctaaaatatatttttagagatggggtagatatattttcagtgatagttcacttagtagaaccatggtttgtaaggtatggtgtttccggagctagttgcaaatagttttctatgtagaacacccttgaaaaatagaagacatctcaaattatttttttaactggtgtgactgtgcgaagaacgctcgtgaggactcacggtcacggcgctggagcacatattggccgccagagtcacccaggtgtacaactccaggaaactaaagttgccacgtcaatccattaccatggcggcggtggcgtggcacagccttttgttttaagataaataaaaaaggaccgaccggcgctgatcttgaccattacaaacttgtttgtttaaaaatattgttcgctgatttattgtgagagaaaaacactgctgaatagctggcagattcaatagataagcttaaccaaatacattagtcactgctctaggtctccaacgtacagacaaaatgcagagaaaatgaagaagaaatgatgagcacgcgactgagagtgaatgaatacactgcagtcatagtacgtttgtaatcagctgtatcaaagttcttgacaaaatatatatataatacagggcttgcacatccaacagtcttggaacagaaactaagattaattagcatggtatataaattaaaggtgcaacaaacatcattgcaaaattaaaagtgacatctcaaaagcacgccacgggtaaagaagaccacacagcgcgcgcgcacaactgtaaatgaagtaatccaaagcaaagcggccatggtaggccgcctaaaacaaagttgacacagcagaataagaccaacgaccatacggactaatcaagcacacatgcatgaagaaaagacaaatccattaaacagcaccagaagaagttgctgcatcgccatcttcttcacctggcctgctgacgatcccggcgtggaagagcaacgcccagagcagcgttatcagctcgccacccctggcaatggcctccgagtgacccttgaggttgtctgatggcgcgacgaacaggatcatctctgaccagaagtcagccagcagcttccacgccatctcctcaccattgattttctccaccagctgctttgccagctccacaccattcttgagcacctcatgcttcgagttctcactcagcaagtggaccagcttttgatactcatcttctggctccgatgaccttgctactgcgtgaccggcgagggcatgctctgcgtccttcttgacagccttgtacaagctcttgctccatgcattgtcttatccggaagtagctcagggcaccaactcactaggtatgcacagtactttgacaagtgactggcaacaatcttataatctgtagagctgggatttggtggagaaccctgttcttgatgatcagctcggtatgggtgtttcacttcaaggatgcatgtggcgatatgccatgtgagtatgatatccgatgtacctttgccattgcaagccccaataaagctttgtccaccaacttggctttgtcgtagagattctgcaccattgctcaaacggtgtccattgatcagatttcttactgcattcaggatgctaaccttcacggccgctggcacctccattttcctcctctgatctgggacacggaggagacggtatacaagaactattggggttgtttttggctgaggtaccaacactgagcactggctcattttttcatcccagtgtttcatcagcttgcatctgcagcgtaacaaactactaaccaattttcgcatgcatagtgaatgtggtgaagagttgcggttgatgtagcagcaaataagggtaacttttgtccagttggagcaaatataggaaaccaagcttctaacctcagctgtcaccgctaacatcagaaccaaaaatattggcaacagatcgaaggaccactctcccatattgatgtgcgacatgtagcttgctaagttcccttgaatacaccataatttacactgaagttgttggcccctcccatgctctaagatcttccgtactatgtttgctatgactagcaaagtaaacaagatacaataaactactgtgaaaagtgataaaatgatatccaagatgggcaggcaacaatttgaataggagattgggagagacgagtaataataatcatgaagaaatgataactcttcctcaatcaccacaaataccctttcatcttctccaaccttgtgcaacaagctccggaaaaagttgcgagtcccagtagagccaccattagtgatcttataccgtgcaaaccgacatcgcaacaacttgaacaaagcaaaggacaagcatatatcttttaactgtggcgttgcaattagaagcatgctatctaactgccaaactctatcaatagtcaccaatccaacatgtttgttcagcattgtttcagagtcatccttgaatgtatacccatgaggatgcttctccacttgttcctccgattctctcataaccaagagtgggggagcatgtgcaccatcacctaccagcggttcatcaacatgctgatgtgctgcttcttgtgcttgtaacttctgcatgtaaacaaaaacaagtcctggattgcgtccgagtgtgaaggacgaccgccgtgccatttcgaatgcataaaatttgagccctattttggagcatagcagagcataaggtgtgatgaagatgaagatgagtcctataatgaagccctctgcggcatagagcttaaggagaccaccggcagcaaggtaaaaagtccaaatcccctgtataagcagctcgggcgggggacctttggattggccttctctgtcatcaacagagactattgtgcttgtattgatcatgactaccagcacaagggatgccagaattacgaagaataccacgtggctaagtgcattgcattgtgcaacgatcgccagctcaccataatcaccataatcctcaccataatcttcatacagtgaggaaaacctgatgtaattagggtggaggctgatggtggaggtgagataggagatgatgggcaagaaaagtgtgttggctccaaggaagacaaagcgagtgaaggggtggtggcggtaccgctgaccataggcgcctatcccaacaagtactacagccaggacaggttgaaccactagcagggcattaacctcccatagctgccgtctaatgctctcgtggacggttgtctggagatccatcgcttgtttggagcaatttccgatcagcttcccattccctccagccatgctcagctgcctgccaaacttcaagtcgttagctagcaccatgcaaggcaaggcacatgcaacctcgtgcaagatagacggtgttgtatgtacgagaaactattaacatgcaagtaatttgaagaaaaaaaataaacgagtggaggaattacatgaattaattaaggtgtggcggtatagcttaggccgagatccagctgactggtgctgcgcaatccagcagcccagccgctcagcgtttcttatggatggtttccttcgtaatggagttttgggcttgctagctactcacgcgtattagagaagggtatatatgggagatgccgagaggaagaaagtagaggttgaacctgacctttggaacgattgggtcatgatcccacggctattacagccaagaatctttgatgttccataagtattagaagttttgaggacatatttagtaggtagaactaatcaaatgattatctttactctctcattttattttctaacacagtacccatttaaatactatattctttatttggttgcaataaattaatctacctattactctatatatatgttttaccatcatttgcactgcgccttggtgggtttaatttatatgtatgtgtccaaatattcggtgtgataggagttaaagtttaccgctagaaatcaaacagggactaagtattgtctcggcgctgctaaaactacaccagggaaaaatcgcaaacctttgaagtttgtatatgtcgttatgcttttagttttaagttttactctagatagttgatgatgcaatctgctgtgaatcctttttaattaatctttaaggaaacagcaacatggatgtatgaactgatgactcaacacagtctattttcatgttcaacaaggaaaatgaaataatgattctcttgtgtttttgaattgtaggagactaggaggattccccgcgtgttgccgcgggtatgaagaaggaatataaatgttagaacgtatagaaatgtgacttgctgacagatattataattacataagttagtagattttaattgtatgtgatagtagattgtctagttggatagcttgtatgttgatagaaataggtagttaggtgtcatagtggattgtcacatgttaaaaaaataggtagtgagattttgctttataaaagtatagtaagattgaaagtcgctgcaacacagcaggaaacccagggtttattttagtctggtcattcactcaaatgtacgtaaattagctaggtaatctgctgtggatcggaatatatactttgctttattcatggaagagcaggtgttctgctaatcttttaaaaaaggaataaacaatgaaaaaagaaaagcagctagtaccttaaatcaagcatcatcatatgcatgcgaggtcccctttattcctcagcgcctgcgtttttctttttctctttgatttccgtcttttttttctttaatttctgcctttgtctatcattattaccttctccattgcacagacgatatgaagcaagtcgagtgactagaatggcacgagctcgagtgacttgtggcgagcgtgtgagagagcgtgtgtttggctcagagtaatacatggagaggccaagtctaaacaacgactatcttcatttctatttaaatttgtctaatcaatgattcccacaccattgccgtccactatacaccacttggtacgtgcggagctattaccaccatggccactaaaccactcagcccaccagggtgtcttggagtcagtggcggagccaggatttgaatcttgggtattcctttgttagtgtctagtgagtcgttattcagctgttgcctctaaccttcgaccgacgtgcgctctcgccggtcgccatgtaagcatagcatagagatagaggtcgcgtccgcgctgcatgtcccgcgctgccaggagcttaggacattgagaaccagcagtaccgccaccacatcatgcctggtaggctagggtgagagatttaggccttgtttagttcgcaaaaattttcaagattcctcgtcacttcgaatctttggttgcatgcatggagcattaaatatagatgaaaataaaaacgaattacacagtttacctgtaatttgtaagatgaatcttttgagcctagttactccttgtttagacaatgtttgtcaaataaaaacgaaagtgctacagtagccaaaaatggaaatttttgccaactaaacaaggccttaggaatgaggattatggaatagtctcccactcatgtccttattatatatgctgccatgtgcatatttccatatttagatccaaaaagtttttcgattggagtaactaggcttaaaagatttgtctcacgatttacacgcaatctgtgcaattagtttttattttcatatatattgaaaagttttttggttttaggatgaactaaacaaggccttatgttggaaaagaatttagaaataaaatatgatttgtactaatatttagtgacatggtcaatatgtatgtggtctgaagataaataattagattctcatttacagaaacaacaaattagtacaaattcctttgaaaagaatgagagagaaattttatttgttaaatatagaaatgctaatatatttgatatacatatatataaatatacctatatatacttagtgcacaaaaagttaggtattcctaggaataccaggaatacctggtgtattcgcccatgcttgaggcatgtttggtaacgcgactctgaatggcctggctggctggcctagtctaggtcagctcgagcctggtcataacggtgcaatctcatatgtttggttgtctattctaacagagacgagctcacttcaaatcgtgtttgattggtagtatactagaatctggtaaccctagacttacaacatggtaatgttacctcctctttgtcatttcatcgcgaatacatggaggtcgtcggcgtcgaggcatggcatggctccgattggcggtgaggcatggctgcatacgacggtgaaccctcctccccatccctctctgcttgcctacaccttagtcaccactagcgtaactcgcgaacgcggagcacactacgccattgttgtggctgctcgacctgatgacaagctcgcagccaccgccagggcagcctctctccatgccgcacctggagcacggcatgggagcaagagtgtctacggccggcggcggcgccgcttctaggagcaacgtgaccgaggcggtggtgtcagaggggcgccgctttcaggagcccatccatcgcgttgcgtggtccccgtgtgtccctagaggaggaggcagtgtggcgggactctgattttcccagatctcatgaccgggtccggctctgccaccattgccgtgacagaggcaatgttgctttgcatagcatccgtgcagaataggctatccagtgactcgtacatgccgatgtcgaagaaggcgaactcatcgcagaagaggagcacatttggctcagagccggaggaggcctatggtgatgcctcgtcttctgtgctcttcgactcagtccttggctgcttcttgtcgccactctagcctcaacatcgagggcgggggctgcgtcatacgccgctggcatgccctaccgtgagatgtccaggagtgcgccagagccagagcgctcattggcctcgtacttcaaggcggcgctgctcttcttcctcttggttgacaaggtcgatgacggtggcgtcggtggtgtgcgtgcttgtgactggctagctagggccccgatggtggcggagaagagaaataggtgagaggccgggaggtggcaagttcgttcactgcacgctccttctcgctccacctggctaggctatggaaaaacaaaaccccccatcattttagttgatcccataactataaatagttgattgcgcaactcgacatgtaagtggagggtcggctcccagggggggagaggatagacttagacaaaccgataagctaacaagtggaacaaccgagtgtcttccaccagcagaaatctccacttttgtgacgctagggagaaccctcaccgctgacaccgtaagctagcactgagcaatcccctaccaaactctcattctccttcattataaaccccctgagatttgggtgctcctgaatacgaagaacaccacactactagacgagggcctcgatagccaaaactaggataaaccgtcatgtctatcttgtgtttttaagtacccaagccaccggagacccacacttcttatattcgacaaacaaccatgatgctttgcagttatgaatccgcgacaactagcacacaaagtagggaagtgtcgaatgtgaattcggctctatggtggccggagccacccgacttgtcgccgaagcaggtttaaatgaaggggctgattgatgattcttcttgggatacaaagttggaccatattgggcccaaaaaatgggaggagagaaagcagtcggtggaaatttagtcttcttcaactcctagaactatcattgtgttctgatttttctgctaaaactataaaaccgcatatcttacccccccccccccccctcatctctcagaatcaattacctccctatcctagttaaaaatggtgataaatgccagataagacttttaaaccataaaaatatctctacggatatttacaggccgtgaaagtagctctaaaagctttcaaaaatagatttaccttttcagaaaatagaaaataaacagagaaatctgtagaattcccaaaatattctaattgatgcataaacacgttttcaaaactttctataggaaacatgagggcaatatcagcagcatatgcatttagcatcatatttgtgttgctttcttgtcgtctgcatctcatatttttgttattggaaattccttagacacatttggacatcaattatggaaaatgttcatatttatttggatattttctctttttcttgagctatacataaatttttggaagctactaaagttaatttccttagctctcaatattttatttgggtttataaatattttaattggggttttcaagtaccaatcttactttaggatttctcccaaaattttagaagcttagacgtagttttcatggttaaaaaccttatcatatttttagaaccaaattgtcctcctgagaagcactgctaacggagcggggaagtaattgaaaagactttgaagagttggggtgggggggggggtgggggggggggttaagatttccatttgtgatgttcgaaaaagaaattggactatgatagtagttcaaggaggattgttttttctgaatttgcgagcgtgttctctgttggcccattaggacaatggagaggcacgagacaatatttgcttggcaacggaagcaggcagcaattggcaatatattggcctcatgacttgatttggctacaaaatgcagaagagaaaactaaaccaacatccagatgcatgtagttttgaaaaaaaaatgcatcggcgctgctggaccgcagaacgccgcttctgccatggtctacagacttcggccacctgtgtcatatgtgaccaactacctgagaccattgatcacattcttctgggctgttgtttcagtagggaagtttggcaaatttgcttcagcaagctgcacctgcagggcttcgttgttgtcaatgaggagcctgtgatgcattggtgggttcacagcaggaaggcagtccctaagcaacttcggtgtggattcgacttgctttttttctagtgggctggctgctttcgaaggagaggaatgcgagaactttcgatgcagtcacctcgacactgttacagctggcaacaagaattatggatgatattgttgtctgggaactggcagggtttaaacccttcttggctcttctaggtttggtgtcttcttagtagtcctctccccgcttcctctttagccatcttggctactgcctgttcctttgctgttgagtgttttggttctatgttcggcaacatatggttaccgtgtaactagctgtatccttcaaagctattcctgcttaatgaaaaacatgcctcccgacgtggtctagaaaaaaaaattctttggcacttactcatcaaacgttattctaccatgcaagtcattcgtacaaaagcttacacatacatggatcatcacacagtatgtggagacagtaaacaatttgtatctgaattattagtaaatcttaagagcttcattacaacattatttcaggcaggacaccaaacagactagttacaattgatacagaagacaagagtaccattatcacatcagcagcgacgacaacatcattcagagtggcacctcatcaaatgaaatgcactcggcatgcagaaacattacctcacaggccgacaggcgtacagcgaggcgacaaagtatgaaataattaaaaccaacgctgattttttgttggcgaaagcataacaaaccaaccctgaattaaaacatgcatgtatgagtagtaggtaataggataagactccattagaccacaccagcggcagaggtagcagcagcagcagcagcagcagcactgttgttctcgcctggcctgctgacgatcccagcatggaagagcagcacccatagcagcgttatcagctcgccgcccctggcgattgcctctttgtgccctttgagattgtcggacggcgcaacatacaggaccatctccgaccagaattcagccaacaacttccacgccgtatcatcaccaccatgtagcaccagcttcgccaattgctctcccagcctcgcgccgtccttgactacttgatgcttcgcttttgtactcagcaattcgaccagctgttggcatttgacttcctgcgtcaatgaatctcctgccgtgcagccggcaaggacacgccgagtgtccttcttgacattctcgtacaggctcctgctccattcgtggtcatcgggaagcagctcagggcaccatgtcacgaggtaagcacagtaccgtgataggtgggtggcaaccattttgtagtcagtgttggaaagtggtggagaaccttgtccttcgtcattcaggtttgggtacctgacctcaagtatacttgtggcgatgtgccatgtgagtatggtataagatgtactcttgttgctgcaagcccagttgaagctttcaccaaggtctcggcagcggcggagacatgctgctccattgctgagctgtcctccatcatctctagtccttcttagcgcttgcatgatgcaaaccttcactactgctggcacactgaccttcctgttctggtctggtaaaaaagggaaaaggtaccagagaagaccaagcggggttgctcttggttgaagcaccaatactgagaactggccaattttctcatcccaatgcctcctcaataacttgcatctgcagcggatcaaaaggccggcacatttctgaatgcaaagagaatgcttcgatgaggaggaggaggcacggtttaggaggcgacacacgagggctactttggtccagttagagcagacgtaggaagccatgtccctcacctcagtaatgaaaactaacactaaaagaaaaagtagtggcaccagaccaaagagccagcttccatacaatcttatgtccaattgtgtttgcaactgattttcagtgcaactgatgaaacacaaaagctgaggtgaactagtatttacagctaaaaggaagctgagccacgttgcagccaagatgcaataagttatgctcaagagtgaaataaagatacccacgatgggcaaccagcacttggaataggagatcggtatggatgtgtaataatagtcatgaacaaaagaaatctcctctgaaatcaaccgaaacaccctgttgtggtctccctccttgagcagcaagctccagaaaaaagtgaacatgtcactcgaggcattagcagtgtggacattgtaccctgcaaaccggcaccgcaacaacttgaagaacgcaaaggacaagcataagtcttgtagtcgctgcagtgttgagaacggaaagaagcaggctgtcatcttccaaacactgtcaatagttaccaggccatctttaccgtgcacagccatgcctgagtcacccttccatacatacccatgagactgcttctccatgtttctttttccctctcccataaccactagtggaggtggaggaggagcatcctcattagttactgctgctgcttctctacggtgacttgtccttgcttgtaccggtggtggttgccgcatgtaggcaaaaataagatgaggattgtttccgagagcaaaggattgccgtgccttttcaaatgcataatatttgaacaccattttggcacatgtgagagcaaaaggtatcccttcaagtccaattataagcacaacaaaagtaataagtccatctctggaatttatgccactattattagtgaagtaggtgacacctaggtaataggtccagaggccctgaacaagcaggtcaaaaggaggacctatgtttctgccttctctgtcatcaacagcaactattgcactggtattgatcatgattatctgaacaagggatgcccatactatgaccacgggagattgaaattctggatcgcatcttgcagccaaccgcccctcatctgtgtctgtgacattgtagtttgatccagcgaccaaggggacagcggtggacatgacaggcaagaacaaggtggtggctccaacaaagatgaacctagttaggcggtggtagcggtaacgttggccaaagatgcctatcccaactatgactcccgccaggatggcgctggcgaccaacagagcgttcacaagccacaacctgtgtctgatgctcttgtggaacaaggacagggattggccatcgcatataatccgggaggagcattgtttgagtgcattgtaggagcagtggtctctagttgaattccctccagccatgattgcccctgctcattgccaaaatatcaaggttatataaacacagattattttgtagcatgctgaaatggaaataatatggactatggaggcatgtacatcttatcttacaattactagttagaggcctgaacggaaacaccatgttaattctcactaggcgcactaggaaaaaagataaatcctagaaatcctcataataatcagaaacatctgaccttgaatttggtagacttgaatcatcatcaccgataaatagcaaatggatatattgaatttagaataaaaattacccaccattgtctttaagaaaaatacataaagtgacccccttattttgtatctaaattaccctcctccgccattacacaagatcattcagaataacctcctaaattggtatatagattacccataactgtcattatgaaggataattcaaatagcccccaaaattgacatctaaatttcaaaaatttgtcattataaaacatactaccaaataaccacataacattgcatctaattacccttcttaaccattatgaaagataatttcaaataaccttctaacgttgtatctaaattcccatcttctataacaaaagatgatttaaaatacccctaaagtttttataagttatccacctttgtgattataaaagataaaataacctcctaagtttgcattcaacctacccatatgtgtcattatgaacgataaatattcgatgtacctcagtcactataaaagtaaacaaaatcatctctcaaatatacatgtaaatacttatatatgttggtatggtacatatagatatttcttgcaatcgattatggagaagccaaaaaacgggttatttaatttcttttactttgaataaaaagaagaaacaactatgatgaactcaaggtagacatttctggaagtaggtgggattaccccttttcattgtacggaccataattatgccaaaatcttcaccccaacaggttgagaggccccatcaccgtcttcatgttttggcagtggaagtggacgattatgcccatcttcatcaaatgttggtatcaaactggccatgaaaatcgataaattttttttttaaaaagtcaaggtcaagtgcgtgcccgccacacccatggcaccggtcgtgtccttacgcccctcgacactgttggctgattcaaaccctgatgccctcgtccacccgatcatagatcaacaccgtagggacgccgcctcaccgttgctgccatagagcctcctctgcccccgctgaggggggggggggggcaggggcgaacctagcaacaagctagtgggtgcaaatgcaccacccaaaatttgaaaaaacagtggttttttctaaaattttaccatatatgcaccacatataacacaagtctatgcacccacaaggcaagtgcaccaccctctaatttgctctagcttcgccactgggggggcgggggacctgtgagcgaagggaagcacccaccgcaatggggaggtgcaccaccactagggtcagcatgcctgttgcaagggcaggcatgggtgtggttagggccaacacatccacgccaagggggtcatcccgtcgagtgtctaccatgtgccgggtggtttggtacaccagatggtagggcgagagcctggggaagggagaaacagagggatagagcctgggagagaggggggaggataacactgagttgagagagagagagatggtaagaggatagagttcagatggtagcaaccttactaaatacattgaggatccaaattttgcttatgtgagggggctcttaacatcggttcttctcaacacgaaccggcagtgagaatgtttactgtttgctcataagatgaactggtggtcaaattcattttcaccaccgtccatgttccattctgaaccaaaaatgattttcaacgttgggtcctttatggacatgtggtgaccaacgcttactacgtgaaaatggtgtaatgaaaactatttctttagtagtgcatgcatag

SEQ ID NO: 7 - DNA, 739 bp, synthetic construct
  misc_feature 1..739: note = Synthetic
  source 1..739: mol_type = other DNA
ccaaaatccatctaccacaacagcgcgccatctctgctcattcagataacttggtcagcagcaacagcggcaatggcgtcgtcctcctcgtcaccgacgaagctgctagcgtgcctcgtcgccgcagctctggcgctggctgccaccgtcgtcgcgccgtgcgcggcgcagaactcgccgcaggactacgtgaacccgcacaacgcggcgcgcgccgacgtcggcgtcggcccggtgtcgtgggacgacacggtggccgcgtacgcgcagagctacgcggcgcagcggcagggcgactgcaagctgatccactccggcggtccctacggcgagaacatcttctggggctccgccggcgccgactggtcggcgtccgacgccgtggcgtcgtgggtttccgagaagcagtactacaaccacgacaccaacagctgcgcggacggcaaggtgtgcgggcactacacgcaggtggtgtggcgtgactccacggccatcggctgcgcccgcgtcgtctgcgacaacaacgccggcgtcttcatcatctgcagctacaacccgccgggcaactacgtcggccagagcccatactagacgtagtagtgtgccgtatgcatgaattgaatacatgcaagtatacgtactggggtcggagtgaaaataaattgttgtcaactttataccatactatgaatgttgataaacataataagtcaataaaatcatgtgattgctgaggcat

SEQ ID NO: 8 - DNA, 20 bp, synthetic construct
  misc_feature 1..20: note = Synthetic
  source 1..20: mol_type = other DNA
aggcacgctagcagcttcgt

SEQ ID NO: 9 - DNA, 20 bp, synthetic construct
  misc_feature 1..20: note = Synthetic
  source 1..20: mol_type = other DNA
tgcgggttcacgtagtcctg

SEQ ID NO: 10 - DNA, 20 bp, synthetic construct
  misc_feature 1..20: note = Synthetic
  source 1..20: mol_type = other DNA
cccggtgtcgtgggacgaca

SEQ ID NO: 11 - DNA, 20 bp, synthetic construct
  misc_feature 1..20: note = Synthetic
  source 1..20: mol_type = other DNA
atccactccggcggtcccta

SEQ ID NO: 12 - DNA, 2769 bp, synthetic construct
  misc_feature 1..2769: note = Synthetic
  source 1..2769: mol_type = other DNA
gggcaatcatggctggagggaattcaactagagaccactgctcctacaatgcactcaaacaatgctcctcccggattatatgcgatggccaatccctgtccttgttccacaagagcatcagacacaggttgtggcttgtgaacgctctgttggtcgccagcgccatcctggcgggagtcatagttgggataggcatctttggccaacgttaccgctaccaccgcctaactaggttcatctttgttggagccaccaccttgttcttgcctgtcatgtccaccgctgtccccttggtcgctggatcaaactacaatgtcacagacacagatgaggggcggttggctgcaagatgcgatccagaatttcaatctcccgtggtcatagtatgggcatcccttgttcagataatcatgatcaataccagtgcaatagttgctgttgatgacagagaaggcagaaacataggtcctccttttgacctgcttgttcagggcctctggacctattacctaggtgtcacctacttcactaataatagtggcataaattccagagatggacttattacttttgttgtgcttataattggacttgaagggataccttttgctctcacatgtgccaaaatggtgttcaaatattatgcatttgaaaaggcacggcaatcctttgctctcggaaacaatcctcatcttatttttgcctacatgcggcaaccaccaccggtacaagcaaggacaagtcaccgtagagaagcagcagcagtaactaatgaggatgctcctcctccacctccactagtggttatgggagagggaaaaagaaacatggagaagcagtctcatgggtatgtatggaagggtgactcaggcatggctgtgcacggtaaagatggcctggtaactattgacagtgtttggaagatgacagcctgcttctttccgttctcaacactgcagcgactacaagacttatgcttgtcctttgcgttcttcaagttgttgcggtgccggtttgcagggtacaatgtccacactgctaatgcctcgagtgacatgttcacttttttctggagcttgctgctcaaggagggagaccacaacagggtgtttcggttgatttcagaggagatttcttttgttcatgactattattacacatccataccgatctcctattccaagtgctggttgcccatcgtgggtatctttatttcactcttgagcataacttattgcatcttggctgcaacgtggctcagcttccttttagctgtaaatactagttcacctcagcttttgtgtttcatcagttgcactgaaaatcagttgcaaacacaattggacataagattgtatggaagctggctctttggtctggtgccactactttttcttttagtgttagttttcattactgaggtgagggacatggcttcctacgtctgctctaactggaccaaagtagccctcgtgtgtcgcctcctaaaccgtgcctcctcctcctcatcgaagcattctctttgcattcagaaatgtgccggccttttgatccgctgcagatgcaagttattgaggaggcattgggatgagaaaattggccagttctcagtattggtgcttcaaccaagagcaaccccgcttggtcttctctggtaccttttcccttttttaccagaccagaacaggaaggtcagtgtgccagcagtagtgaaggtttgcatcatgcaagcgctaagaaggactagagatgatggaggacagctcagcaatggagcagcatgtctccgccgctgccgagaccttggtgaaagcttcaactgggcttgcagcaacaagagtacatcttataccatactcacatggcacatcgccacaagtatacttgaggtcaggtacccaaacctgaatgacgaaggacaaggttctccaccactttccaacactgactacaaaatggttgccacccacctatcacggtactgtgcttacctcgtgacatggtgccctgagctgcttcccgatgaccacgaatggagcaggagcctgtacgagaatgtcaagaaggacactcggcgtgtccttgccggctgcacggcaggagattcattgacgcaggaagtcaaatgccaacagctggtcgaattgctgagtacaaaagcgaagcatcaagtagtcaaggacggcgcgaggctgggagagcaattggcgaagctggtgctacatggtggtgatgatacggcgtggaagttgttggctgaattctggtcggagatggtcctgtatgttgcgccgtccgacaatctcaaagggcacaaagaggcaatcgccaggggcggcgagctgataacgctgctatgggtgctgctcttccatgctgggatcgtcagcaggccaggcgagaacaacagtgctgctgctgctgctgctgctgctacctctgccgctggtgtggtctaatggagtcttatcctattacctactactcatacatgcatgttttaattcagggttggtttgttatgctttcgccaacaaaaaatcagcgttggttttaattatttcatactttgtcgcctcgctgtacgcctgtcggcctgtgaggtaatgtttctgcatgccgagtgcatttcatttgatgaggtgccactctgaatgatgttgtcgtcgctgctgatgtgataatggtactcttgtcttctgtatcaattgtaactagtctgtttggtgtcctgcctgaaataatgttgtaatgaagctcttaagattt

SEQ ID NO: 13 - DNA, 20 bp, synthetic construct
  misc_feature 1..20: note = Synthetic
  source 1..20: mol_type = other DNA
ttggccatcgcatataatcc

SEQ ID NO: 14 - DNA, 20 bp, synthetic construct
  misc_feature 1..20: note = Synthetic
  source 1..20: mol_type = other DNA
tcaaccgaaacaccctgttg

SEQ ID NO: 15 - DNA, 20 bp, synthetic construct
  misc_feature 1..20: note = Synthetic
  source 1..20: mol_type = other DNA
aatctcccgtggtcatagta

SEQ ID NO: 16 - DNA, 20 bp, synthetic construct
  misc_feature 1..20: note = Synthetic
  source 1..20: mol_type = other DNA
gtgcttataattggacttga

SEQ ID NO: 17 - DNA, 2654 bp, synthetic construct
  misc_feature 1..2654: note = Synthetic
  source 1..2654: mol_type = other DNA
gcctgcgaagcccaagaaactaaagcaggcgcctgcgttagcctgcaaggcttgtgcgtgggaagcacaccggcgccgcatcggccacatgagcggactcctactcgagcgccatatccgcagcggcggcgctagggaagccggtgagaatcactcatctgctgtcggtgagaatcactcatctactgttcttcttctcccttgttcttctaatctgaatcttcttgtttttgttggtgtcactagtcaccctggacgccgccctacctccagatggacgcgaggcgtggagccgcgaggcctccaccgtcggcacccgcgccacggcgcgcgggccgccgagcacacagaggatgtccgcggctacgccctctagggagctggctgatgtgcggggtgaggaggaggaggaggcgcagcaggagacagagaaggagagggtgaagaccagcttcaacgacaggtgagctgtctcctgcctcctctgacgctctgacgcttctgttgcttgtgctatcgggaagaaaaaagaaggggggaggaaagccgagtgccagcgcaccactcatcgccggccaacttagccttgataaataagctagttcatctgcgtctcggtttctccccaatcccaacttggatggggatagatgattcacatactgttgtttgtgtctgttctaacggggttggtaaatttaccggatatagcctttattcatagcacccacgtaaaagaaaaaaacagaaaaaggatagctatactgcttcgggagttctggctcacaattttggttgagaacatgtacatttctgagccaggcaactcgttggtactaatgtctgaatttttgtcgattgctaagaaatcagaggtctcatagcttgttggtattcgaaggtttgtctggaaccgtcagttgcttattattcagaggttcaactactgcagtactgcttcctgttaactctgatgacagtttttatctgattgggatttgtgaagcgttaagtttaatctcatgcttcgtatatcttctgagttgcaatgcttcctcttgacaagagtcgaaaccgctttggatggcctgagtcgctgtgatcgtgaggtgcgtgaatacagacaaagaggaccttgttggcgtataaatgttttagtatatcgtccgcatcgcgctgtcccgcgacggtaggctgctcgccatcaagcatatatatgttttagtatcattttttcacattgcattatgcccagatacttgctcacatatattgcagtgatgtttttttccttctatgttgtgatgtttttttctatttttttaactcaattcaatgactgatattatctcattttatactttagctctgttaaaatgacagccatgttacagcctgaacaagaagttggggcttcttcaaagaaatcaacattgattagctcttccatcacaaagcgtaaaaggccttcatccaaggatcaacaaacatactccactgtagcaaaaaagtcaaaggctcctatccttccctaacctcattctgtagcaggagagcagcaaagccctgatccactaggctcgcacactatcaggtcgtcgtcactcctccacgtcctgcccccacggtaagattagtgttgcctctccggccacctctccatttaggttgtagatggatttttgattcttagatgattcatattatctctttttatacttcagccctgttaccatggcaacaatgacaatcatatcacagtctgaaccagaaggtggaggttcttcaaagaaatcagcattggttagctcttcaagcacaaagcataaaaggtcaaagacatctacatcggcaaaacaacagaactctccttcaacatctccattgaagaggaggccttcggaaaaaaatcaacaaaaacaagctgcaaagagatttaagcctccaatggtgacaaggtctcccatggttacaaggtctccaataattatgttaatggtgtctcctatggtgattgggcagaggtcttcacatagtccaaggagccctccagtattatcatcctcaccccctgttgcatcaccaccacagtcaaacgcacatgattcgggtaattgtcattgctttctgttcttatgtatttgtttagcaataagttgtgttattatttcagcacataatttcttacttacatgtaactaagctaatcggaattttttttacattaatgatacagattccactttggcacatagtaaaccacgcaagttaagatctgagatttggattgatatggagccaatttatcatcaaggaaaggttatacaagctcgatgcaagcactgcaatgaggtattctctgcaacacgtaattgtggcaatagtcacatgttagagtcagctttgccattcaaggatgcttttcaagagttaggccacctagatcccaactacatttactcttgcgcacgaagtacttttcccgagattcaggaatggctgtagaatctagtctggatttcctctcgcctcttgtgtaactaataagttttttttttctccttttgcttctttgtttcttaccctcagctctgtaaataacttttttcctttgataattggttgcagtaggggtacggtactcctcgaagagc

SEQ ID NO: 18 - DNA, 20 bp, synthetic construct
  misc_feature 1..20: note = Synthetic
  source 1..20: mol_type = other DNA
aaaacatttatacgccaaca

SEQ ID NO: 19 - DNA, 20 bp, synthetic construct
  misc_feature 1..20: note = Synthetic
  source 1..20: mol_type = other DNA
tccgaaggcctcctcttcaa

SEQ ID NO: 20 - DNA, 20 bp, synthetic construct
  misc_feature 1..20: note = Synthetic
  source 1..20: mol_type = other DNA
tcacagtctgaaccagaagg

SEQ ID NO: 21 - DNA, 20 bp, synthetic construct
  misc_feature 1..20: note = Synthetic
  source 1..20: mol_type = other DNA
tatgttaatggtgtctccta

SEQ ID NO: 22 - DNA, 53 bp, synthetic construct
  source 1..53: mol_type = other DNA
gctcataagatgaactggtggtcaaattcattttcaccaccgtccatgttcca